(19) United States
(12) Patent Application Publication
(10) Pub. No.: US 2026/0036559 A1
(43) Pub. Date: Feb. 5, 2026

(54) Harmful Gas Detection Device

(71) Applicant: Guizhou Power Grid Co., Ltd., Guiyang (CN)

(72) Inventors: Chaoping XU, Guiyang (CN); Xi FANG, Guiyang (CN); Jialong WU, Guiyang (CN); Lei HUANG, Guiyang (CN); Ying LIU, Guiyang (CN); Xufei YANG, Guiyang (CN); Shi WANG, Guiyang (CN); Qun YU, Guiyang (CN); Wei WU, Guiyang (CN); Xin TENG, Guiyang (CN); Xinxin WANG, Guiyang (CN); Chonglin XIANG, Guiyang (CN); Yudong HUANG, Guiyang (CN); Jiawei XU, Guiyang (CN); Yuxiong XIAO, Guiyang (CN); Junwen LONG, Guiyang (CN); Qian XIAO, Guiyang (CN); Ruiyang WU, Guiyang (CN); Fengyi DENG, Guiyang (CN); Xiangyue DAI, Guiyang (CN); Dan JIANG, Guiyang (CN); Yuan HUANG, Guiyang (CN); Siyuan CHEN, Guiyang (CN); Hailu ZHANG, Guiyang (CN); Changyu TANG, Guiyang (CN)

(21) Appl. No.: 19/170,521

(22) Filed: Apr. 4, 2025

(30) Foreign Application Priority Data
Aug. 1, 2024 (CN) 202411050821.6

(51) Int. Cl. G01N 33/00

(52) U.S. Cl. CPC G01N 33/0027; G01N 33/0063

(57) ABSTRACT

Disclosed is a harmful gas detection device, comprising a hand-held mechanism, a detection mechanism, and an external mechanism, where the detection mechanism comprises a connecting cylinder, a detector body, and interference components; and the external mechanism comprises a wheel body, an external sleeve, and a rotating component. The detection mechanism can be placed in front of a staff by the hand-held mechanism, and the detection mechanism can introduce air from different positions on an advancing road into the detector body during movement. The wheel body can increase the load capacity, which can better improve the battery storage capacity.

### CROSS-REFERENCE TO RELATED APPLICATIONS

The present application claims priority to Chinese Patent Application No. 202411050821.6, filed on Aug. 1, 2024, the entire disclosure of which is incorporated herein by reference.

### TECHNICAL FIELD

The present invention relates to the technical field of underground comprehensive pipe galleries, in particular to a harmful gas detection device.

### BACKGROUND

The underground comprehensive pipe gallery is a common pipeline for various projects, built underground in the city and used as a public tunnel for centralized laying of municipal pipelines such as electricity, communication, broadcasting and television, and water supply. It is equivalent to the lifeline of modern urban operation. Once problems arise, even the entire city may be affected. As the population size and urban demand increase, the scale of the underground comprehensive pipe gallery is increasingly large, and its responsibilities are increasingly important. When the scale of the underground comprehensive pipe gallery expands, the maintenance of electrical equipment and the safety of personnel in the pipe gallery are increasingly valued.

When entering the pipe gallery, the staff needs to carry corresponding detection equipment, usually a pump suction gas detector. However, in the actual work process, when the detection equipment worn on the staff detects harmful gases, the staff is already in a relatively dangerous area. Therefore, a device capable of exploratory detection, added with multi-directional air suction inside, is designed to improve efficiency.

### SUMMARY

The objective of this section is to outline some aspects of the embodiments of the present invention and briefly introduce some preferred embodiments. Some simplifications or omissions may be made in this section, as well as in the abstract of the specification and the title of the present invention, to avoid blurring the objectives of this section, the abstract of the specification, and the title of the present invention, and such simplifications or omissions cannot be used for limiting the scope of the present invention.

In view of the problem in the above or existing technologies that harmful gas detection devices cannot detect the presence of harmful gases in front environments in a timely manner, the present invention is proposed.

Therefore, the objective of the present invention is to provide a harmful gas detection device.

To solve the above technical problems, the present invention provides the following technical solution: A harmful gas detection device includes a hand-held mechanism, a detection mechanism arranged on the hand-held mechanism, and an external mechanism arranged on the detection mechanism; the detection mechanism includes a connecting cylinder arranged on the hand-held mechanism, a detector body arranged inside the connecting cylinder, and interference components arranged at two ends of the connecting cylinder; and the external mechanism includes a wheel body and an external sleeve arranged on the connecting cylinder, a rotating component arranged on the external sleeve, an interlacing component arranged on the external sleeve, and a sleeve ring arranged on the other side of the wheel body.

As a preferred solution for the harmful gas detection device according to the present invention, a plurality of air inlet holes are provided at the two ends of the connecting cylinder, an isolation mesh is provided inside the air inlet holes, and an external groove is provided on the side of the outer wall of the connecting cylinder near the air inlet holes.

As a preferred solution for the harmful gas detection device according to the present invention, the interference component includes a rotating ring arranged on the inner wall of the connecting cylinder, embedding grooves are provided at connections between the rotating ring and the connecting cylinder, the connecting cylinder is located in the embedding grooves and fixedly connected to an external plate, a reset spring is provided on the external plate, a connecting plate is provided between the other end of the reset spring and the rotating ring, and a mesh grid is provided on an inner wall of the rotating ring.

As a preferred solution for the harmful gas detection device according to the present invention, the reset spring is arc-shaped, the reset spring is located in the embedding grooves, and the rotating ring is rotatably connected to the connecting cylinder.

As a preferred solution for the harmful gas detection device according to the present invention, the rotating component includes a circular plate on the external sleeve, an internal groove arranged on the circular plate, a contraction spring arranged on the internal groove, and a docking block arranged on the contraction spring; the docking block is provided with a first inclined surface.

As a preferred solution for the harmful gas detection device according to the present invention, the rotating component further includes a driving rod arranged on the rotating ring and a second inclined surface arranged on the driving rod, the cross-section of the internal groove is rectangular, the quantity of the internal groove is plural, the plurality of internal grooves are distributed in a circular array around the circular plate, and the driving rod corresponds one-to-one with the docking block.

As a preferred solution for the harmful gas detection device according to the present invention, a connecting pipe is provided at one end of the circular plate away from the rotating ring, the other end of the connecting pipe is fixedly connected to an arc block, and the connecting pipe penetrates through the arc block.

As a preferred solution for the harmful gas detection device according to the present invention, the interlacing component includes a contact cover arranged on one side of the arc block, a plurality of air inlet pipes are provided on the contact cover, a guide box is provided at a tail end of the air inlet pipe, and the plurality of air inlet pipes are connected by a fixing ring; a sealing ring attached to the external groove is provided on the sleeve ring, and a notch is provided on the sleeve ring.

As a preferred solution for the harmful gas detection device according to the present invention, the plurality of air inlet pipes are arranged in a circular array on the contact cover, and the notch is adapted to the air inlet holes.

As a preferred solution for the harmful gas detection device according to the present invention, the hand-held mechanism includes a support rod arranged on the connecting cylinder, a handle arranged at the other end of the support rod, and a folding support leg arranged at a bottom of the support rod.

Beneficial effects of the harmful gas detection device according to the present invention are as follows: The detection mechanism can be placed in front of a staff by the hand-held mechanism, and the detection mechanism can introduce air from different positions on an advancing road into the detector body during movement, thereby improving the detection effect. The wheel body can effectively increase the load capacity, which can better improve the battery storage capacity of the device, thereby increasing the operating time of the device.

### BRIEF DESCRIPTION OF DRAWINGS

To describe the technical solutions in embodiments of the present invention more clearly, the following briefly describes the accompanying drawings required for describing the embodiments. Apparently, the accompanying drawings in the following descriptions show merely some embodiments of the present invention, and those of ordinary skill in the art may derive other drawings from these accompanying drawings without creative efforts.

FIG. 1 is an overall schematic diagram of a harmful gas detection device.

FIG. 2 is a schematic diagram of a bottom structure of a casing body of the harmful gas detection device.

FIG. 3 is a schematic diagram of a middle structure of the casing body of the harmful gas detection device.

FIG. 4 is a schematic structural diagram of a sealing mechanism of the harmful gas detection device.

FIG. 5 is a schematic structural diagram of a fixing component of the harmful gas detection device.

FIG. 6 is a cross-sectional view of the fixing component of the harmful gas detection device.

FIG. 7 is a schematic structural diagram of a positioning mechanism of the harmful gas detection device.

### DETAILED DESCRIPTION OF THE EMBODIMENTS

In order to make the above objectives, features and advantages of the present invention more obvious and understandable, specific embodiments of the present invention will be described in detail below with reference to the accompanying drawings of the specification.

Many specific details are set forth in the following description to facilitate a full understanding of the present invention, but the present invention can also be implemented in other ways different from those described here, and those skilled in the art can do similar promotions without departing from the connotation of the present invention. Therefore, the present invention is not limited by the specific embodiments disclosed below.

Secondly, the term “one embodiment” or “embodiments” referred to here refers to specific features, structures, or features that may be included in at least one implementation of the present invention. The term “in one embodiment” appearing in different places of this specification neither necessarily refers to the same embodiment, nor is a separate or selective embodiment exclusive with other embodiments.

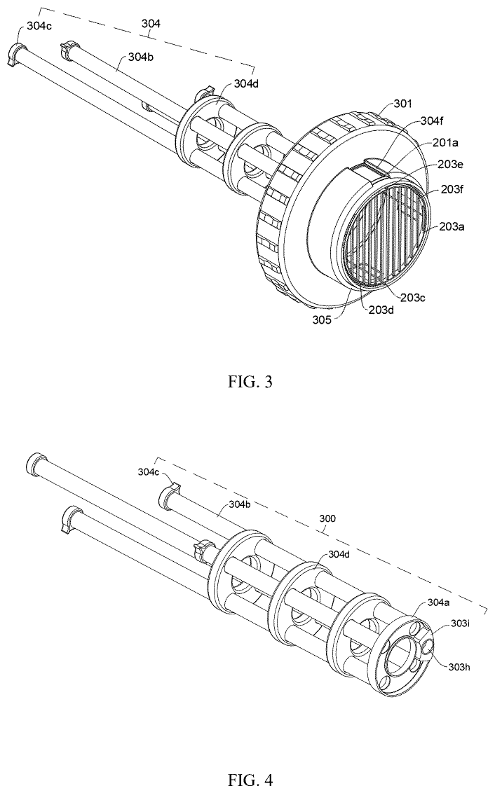

Embodiment 1 FIG. 1 to FIG. 3 show a first embodiment of the present invention. This embodiment provides a harmful gas detection device, including a hand-held mechanism 100, a detection mechanism 200 arranged on the hand-held mechanism 100, and an external mechanism 300 arranged on the detection mechanism 200; the detection mechanism 200 includes a connecting cylinder 201 arranged on the hand-held mechanism 100, a detector body 202 arranged inside the connecting cylinder 201, and interference components 203 arranged at two ends of the connecting cylinder 201.

Specifically, the hand-held mechanism 100 can effectively drive the detection mechanism 200 to move and drive the connecting cylinder 201 to move. The connecting cylinder 201 is fixedly connected to the detector body 202 in the detection mechanism 200. The detector body 202 is a pump suction gas detector with an internal power supply. A wheel body 301 can effectively increase the load capacity of the device to increase power reserve, thereby prolonging the service time of the device. The interference components 203 can effectively interfere with a rotating component 303, thereby blocking the rotation of the rotating component 303. The blocked rotating component 303 can effectively adjust the position between a contact cover 304a and an arc block 303i.

A plurality of air inlet holes 201a are provided at the two ends of the connecting cylinder 201, an isolation mesh 201b is provided inside the air inlet holes 201a, and an external groove 201c is provided on the side of the outer wall of the connecting cylinder 201 near the air inlet holes 201a.

The hand-held mechanism 100 includes a support rod 101 arranged on the connecting cylinder 201, a handle 102 arranged at the other end of the support rod 101, and a folding support leg 103 arranged at a bottom of the support rod 101.

Preferably, the cross-section of the air inlet holes 201a is rectangular. The air inlet holes 201a can effectively limit the isolation mesh 201b, which can effectively introduce external air into the connecting cylinder 201. The isolation mesh 201b can effectively filter the air entering the connecting cylinder 201. The cross-section of the isolation mesh 201b is rectangular, and the isolation mesh 201b is located in a middle area of the air inlet holes 201a.

In summary, the wheel body 301 can effectively increase the load capacity, which can better improve the battery storage capacity of the device, thereby increasing the operating time of the device and ensuring stability during operation and maintenance.

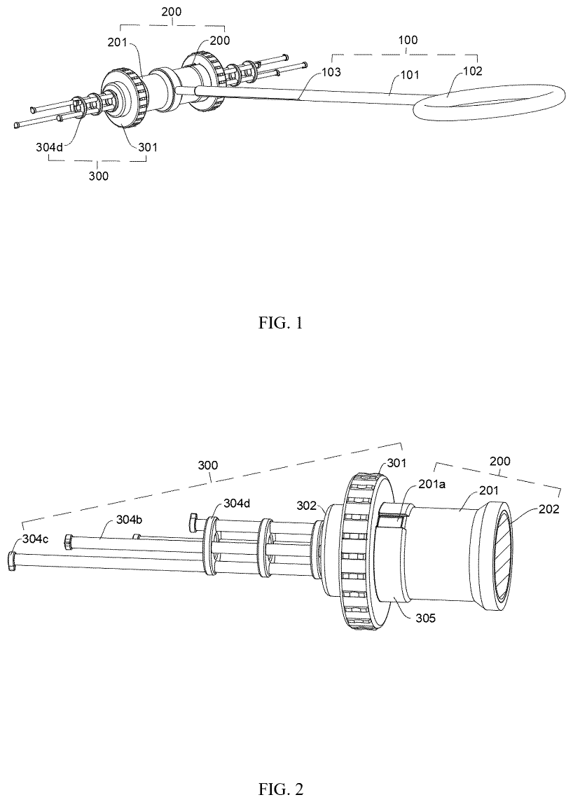

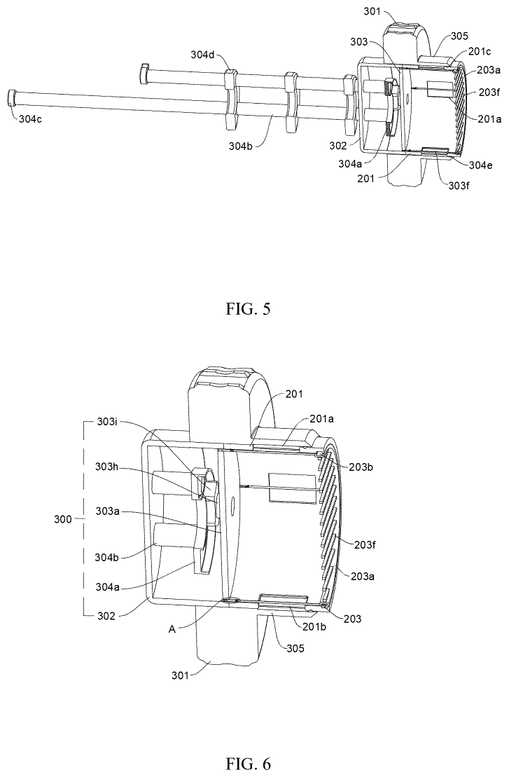

Embodiment 2 FIGS. 2-5 show a second embodiment of the present invention. Unlike the previous embodiment,

The interference component 203 includes a rotating ring 203a arranged on the inner wall of the connecting cylinder 201, embedding grooves 203b are provided at connections between the rotating ring 203a and the connecting cylinder 201, the connecting cylinder 201 is located in the embedding grooves 203b and fixedly connected to an external plate 203c, a reset spring 203d is provided on the external plate 203c, a connecting plate 203e is provided between the other end of the reset spring 203d and the rotating ring 203a, and a mesh grid 203f is provided on an inner wall of the rotating ring 203a.

Specifically, the cross-section of the rotating ring 203a is circular, and the rotating ring 203a is made of stainless steel. The rotating ring 203a can effectively limit the reset spring 203d, and the reset spring 203d can effectively drive the rotating ring 203a to reset. The rotating ring 203a can be used for effectively fixing the external plate 203c. The cross-section of the rotating ring 203a is rectangular, the rotating ring 203a is fixedly connected to the external plate 203c, and the external plate 203c is fixedly connected to the reset spring 203d. The connecting plate 203e can effectively limit the reset spring 203d. The reset spring 203d is fixedly connected to the connecting plate 203e, and the connecting plate 203e is fixedly connected to the connecting cylinder 201. The connecting cylinder 201 does not rotate during operation, but only the wheel body 301 and an external sleeve 302 rotate during the use of the device. The mesh grid 203f can effectively protect a main body of the detection mechanism 200.

The reset spring 203d is arc-shaped, the reset spring 203d is located in the embedding grooves 203b, and the rotating ring 203a is rotatably connected to the connecting cylinder 201.

Further, the cross-section of the embedding groove 203b is circular, an inner wall of the embedding groove 203b is attached to an outer wall of the reset spring 203d, and the connecting cylinder 201 is made of stainless steel. The connecting cylinder 201 can effectively limit the rotating ring 203a.

The remaining structures are the same as in Embodiment 1.

In summary, the rotating ring 203a can effectively limit the reset spring 203d, the reset spring 203d can effectively drive the rotating ring 203a to reset, the rotating ring 203a can be used for effectively fixing the external plate 203c, and the connecting plate 203e can effectively limit the reset spring 203d.

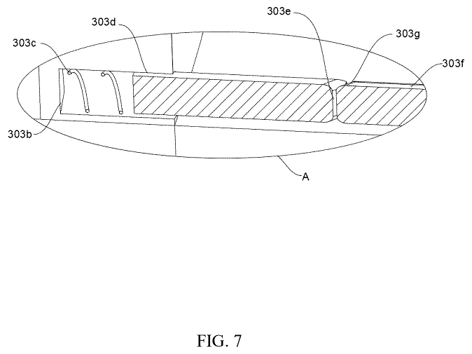

Embodiment 3 FIGS. 1-7 show a third embodiment of the present invention. The external mechanism 300 is unlike the previous embodiment. The external mechanism 300 includes a wheel body 301 and an external sleeve 302 arranged on the connecting cylinder 201, a rotating component 303 arranged on the external sleeve 302, an interlacing component 304 arranged on the external sleeve 302, and a sleeve ring 305 arranged on the other side of the wheel body 301. The rotating component 303 includes a circular plate 303a on the external sleeve 302.

Specifically, the external mechanism 300 can effectively support the connecting cylinder 201, ensure the distance between the connecting cylinder 201 and the ground, and prevent air from entering the connecting cylinder 201 and sucking dust to affect the detection effect of the detector body 202. The rotating component 303 is arranged on two sides of the connecting cylinder 201 and can effectively follow the movement of the connecting cylinder 201. During operation, the connecting cylinder 201 moves in a translational state, and the rotating component 303 moves in a rotational state. The interlacing component 304 can effectively block the rotating component 303. During operation, the position between the contact cover 304a and the arc block 303i can be changed, thereby continuously changing the connection between the arc block 303i and different air inlet pipes 304b. so that a connecting pipe 303h in the arc block 303i alternately contacts the plurality of air inlet pipes 304b. The arc block 303i and the contact cover 304a are both made of rubber, which can achieve the required degree of sealing between the arc block 303i and the contact cover 304a.

The rotating component includes an internal groove 303b arranged on the circular plate 303a, a contraction spring 303c arranged on the internal groove 303b, and a docking block 303d arranged on the contraction spring 303c. The docking block 303d is provided with a first inclined surface 303e. The rotating component 303 further includes a driving rod 303f arranged on the rotating ring 203a and a second inclined surface 303g arranged on the driving rod 303f. The cross-section of the internal groove 303b is rectangular. The quantity of the internal groove 303b is plural, the plurality of internal grooves 303b are distributed in a circular array around the circular plate 303a, and the driving rod 303f corresponds one-to-one with the docking block 303d.

Further, the rectangular internal groove 303b facilitates better contact with the docking block 303d, which can effectively prevent the rotation of the driving rod 303f during the rotation of the circular plate 303a. Two ends of the contraction spring 303c are fixedly connected to the docking block 303d and the internal groove respectively, and the first inclined surface 303e is attached to the second inclined surface 303g. The cross-section of the driving rod 303f is rectangular, the driving rod 303f is made of stainless steel, and the driving rod 303f is fixedly connected to the rotating ring 203a. The internal groove 303b facilitates better limiting of the contraction spring 303c.

A connecting pipe 303h is provided at one end of the circular plate 303a away from the rotating ring 203a, the other end of the connecting pipe 303h is fixedly connected to the arc block 303i, and the connecting pipe 303h penetrates through the arc block 303i. The interlacing component 304 includes a contact cover 304a arranged on one side of the arc block 303i, a plurality of air inlet pipes 304b are provided on the contact cover 304a, a guide box 304c is provided at a tail end of the air inlet pipe 304b, and the plurality of air inlet pipes 304b are connected by a fixing ring 304d;

Furthermore, the arc block 303i is fixedly connected to the connecting pipe 303h, the plurality of air inlet pipes 304b are distributed in a circular array around the fixing ring 304d, an air inlet end of the guide box 304c faces outward, and the plurality of air inlet pipes 304b have different lengths. The arc block 303i facilitates better connection between the contact cover 304a and the connecting pipe 303h, and the fixing ring 304d facilitates better limiting of the air inlet pipes 304b. During collision in operation, the fixing ring 304d can provide protection.

A sealing ring 304e attached to the external groove 201c is provided on the sleeve ring 305, and a notch 304f is provided on the sleeve ring 305. The plurality of air inlet pipes 304b are arranged in a circular array on the contact cover 304a, and the notch 304f is adapted to the air inlet holes 201a.

Preferably, the sleeve ring 305 can effectively block the air inlet holes 201a, and the notch 304f on the sleeve ring 305 can effectively connect the air inlet holes 201a with external air, making it convenient to introduce the external air into the detector body 202 during operation. When one of the two notches 304f on the two contact covers 304a is completely in communication with the air inlet holes 201a during operation, the other notch 304f is interlaced with the air inlet holes 201a, thereby ensuring the suction force of the detector body 202 during operation and better introducing gas in the external air into the detector body 202.

The remaining structures are the same as in Embodiment 2.

During use, the device is placed in user's advancing direction, the user holds the handle 102 and pushes the device forward, the handle 102 moves and drives the connecting cylinder 201 to move, the detector body 202 is activated, and the detector body 202 sucks external air and detects harmful gases in the air. When harmful gases appear, an alarm is given;

When the connecting cylinder 201 moves, gravities produced by the connecting cylinder 201 and components connected thereto are applied to the wheel body 301. Under the action of gravities, friction is produced between the wheel body 301 and the ground to drive the wheel body 301 to rotate. The external sleeve 302 is driven to rotate during the rotation of the wheel body 301, the air inlet pipes 304b are driven to rotate during the rotation of the external sleeve 302, the arc block 303i is driven to rotate during the rotation of the air inlet pipes 304b, the circular plate 303a is driven to rotate during the rotation of the arc block 303i, and the docking block 303d is driven to rotate during the rotation of the circular plate 303a. After the docking block 303d comes into contact with the driving rod 303f, the first inclined surface 303e is in contact with the second inclined surface 303g, which increases the rotation resistance to the circular plate 303a. As a result, the arc block 303i rotates on the contact cover 304a, the arc block 303i alternately contacts the plurality of air inlet pipes 304b, and the detector body 202 suck external air from different air inlet pipes 304b;

Meanwhile, the sleeve ring 305 is driven to rotate during the rotation of the wheel body 301, and the notch 304f is driven to rotate during the rotation of the sleeve ring 305. When the notch 304f rotates, air at different heights is sequentially sucked via the air inlet holes 201a, thereby improving the detection effect;

When the device is placed, the folding support leg 103 is laid down, the ground and the folding support leg 103 form a support, and the external mechanism 300 and the detection mechanism 200 are in contact with the ground to form a triangular area, thereby ensuring the stability of the device.

In summary, the sleeve ring 305 can effectively block the air inlet holes 201a, and the external mechanism 300 can effectively support the connecting cylinder 201, thereby ensuring the distance between the connecting cylinder 201 and the ground and avoiding sucking dust when air enters the connecting cylinder 201.

It is important to note that the construction and arrangement of the present application shown in multiple different exemplary embodiments are only illustrative. Although only several embodiments are described in detail in the disclosure, those who refer to the disclosure should easily understand that many modifications are possible (such as the size, scale, structure, shape and proportion of various elements, parameter values (such as temperature and pressure), installation and arrangement, use of materials, colors, and directional changes), without deviating from the novel teachings and advantages of the subject matter described in the application. For example, elements shown as integrally formed may be composed of multiple portions or elements, positions of the elements may be inverted or otherwise changed, and the properties or number or position of discrete elements may be changed or altered. Therefore, all such modifications are intended to fall within the scope of the present invention. The order or sequence of any process or method step may be changed or reordered according to alternative embodiments. In the claims, any provision of “device plus function” is intended to cover the structure described herein that performs the function, and is not only structurally equivalent but also equivalent. Other substitutions, modifications, changes, and omissions may be made in the design, operation status, and arrangement of the exemplary embodiments without departing from the scope of the present invention. Therefore, the present invention is not limited to specific embodiments, but extends to various modifications that still fall within the scope of the appended claims.

Moreover, in order to provide concise descriptions of the exemplary embodiments, all features (namely, those features unrelated to the currently considered optimal mode of executing the present invention, or those features unrelated to the implementation of the present invention) of the actual embodiments may not be described.

It should be understood that in the development process of any practical implementation, such as in any engineering or design project, a large number of specific implementation decisions can be made. Such development efforts may be complex and time-consuming, but for ordinary technical personnel who benefit from the disclosure, excessive experimentation is not required, and the development efforts will be a routine task of design, manufacturing, and production.

It should be noted that the above embodiments are merely used for illustrating, but not limiting, the technical solutions of the present invention. Although the present invention is described in detail with reference to preferred embodiments, those of ordinary skill in the art should understand that the technical solutions of the present invention can be modified or equivalently substituted without departing from the spirit and scope of the technical solutions of the present invention, and all the modifications and equivalent substitutions should fall within the scope of the claims of the present invention.

What is claimed is:

1. A harmful gas detection device, comprising a hand-held mechanism, a detection mechanism arranged on the hand-held mechanism, and an external mechanism arranged on the detection mechanism, wherein the detection mechanism comprises a connecting cylinder arranged on the hand-held mechanism, a detector body arranged inside the connecting cylinder, and interference components arranged at two ends of the connecting cylinder; and
the external mechanism comprises a wheel body and an external sleeve arranged on the connecting cylinder, a rotating component arranged on the external sleeve, an interlacing component arranged on the external sleeve, and a sleeve ring arranged on the other side of the wheel body.

2. The harmful gas detection device according to claim 1, wherein a plurality of air inlet holes are provided at the two ends of the connecting cylinder, an isolation mesh is provided inside the air inlet holes, and an external groove is provided on the side of the outer wall of the connecting cylinder near the air inlet holes.

3. The harmful gas detection device according to claim 2, wherein the interference component comprises a rotating ring arranged on the inner wall of the connecting cylinder, embedding grooves are provided at connections between the rotating ring and the connecting cylinder, the connecting cylinder is located in the embedding grooves and fixedly connected to an external plate, a reset spring is provided on the external plate, a connecting plate is provided between the other end of the reset spring and the rotating ring, and a mesh grid is provided on an inner wall of the rotating ring.

4. The harmful gas detection device according to claim 3, wherein the reset spring is arc-shaped, the reset spring is located in the embedding grooves, and the rotating ring is rotatably connected to the connecting cylinder.

5. The harmful gas detection device according to claim 4, wherein the rotating component comprises a circular plate on the external sleeve, an internal groove arranged on the circular plate, a contraction spring arranged on the internal groove, and a docking block arranged on the contraction spring; the docking block is provided with a first inclined surface.

6. The harmful gas detection device according to claim 5, wherein the rotating component further comprises a driving rod arranged on the rotating ring and a second inclined surface arranged on the driving rod, the cross-section of the internal groove is rectangular, the quantity of the internal groove is plural, the plurality of internal grooves are distributed in a circular array around the circular plate, and the driving rod corresponds one-to-one with the docking block.

7. The harmful gas detection device according to claim 6, wherein a connecting pipe is provided at one end of the circular plate away from the rotating ring, the other end of the connecting pipe is fixedly connected to an arc block, and the connecting pipe penetrates through the arc block.

8. The harmful gas detection device according to claim 7, wherein the interlacing component comprises a contact cover arranged on one side of the arc block, a plurality of air inlet pipes are provided on the contact cover, a guide box is provided at a tail end of the air inlet pipe, and the plurality of air inlet pipes are connected by a fixing ring; and
a sealing ring attached to the external groove is provided on the sleeve ring, and a notch is provided on the sleeve ring.

9. The harmful gas detection device according to claim 8, wherein the plurality of air inlet pipes are arranged in a circular array on the contact cover, and the notch is adapted to the air inlet holes.

10. The harmful gas detection device according to claim 9, wherein the hand-held mechanism comprises a support rod arranged on the connecting cylinder, a handle arranged at the other end of the support rod, and a folding support leg arranged at a bottom of the support rod.
